(19) United States
(12) Patent Application Publication
(10) Pub. No.: US 2025/0221727 A1
(43) Pub. Date: Jul. 10, 2025

(54) MULTI-FUNCTION ULTRASONIC BLADES AND SURGICAL INSTRUMENTS INCORPORATING THE SAME

(71) Applicant: Covidien LP, Mansfield, MA (US)

(72) Inventor: Matthew S. COWLEY, Boulder, CO (US)

(73) Assignee: Covidien LP, Mansfield, MA (US)

(21) Appl. No.: 18/727,770

(22) PCT Filed: Jan. 13, 2023
(86) PCT No.: PCT/IB2023/050326

§ 371 Date: Jul. 10, 2024

Related U.S. Application Data
(60) Provisional application No. 62/299,637, filed on Feb. 25, 2016.

(51) Int. Cl. A61B 17/32

(52) U.S. Cl. CPC A61B 17/320068; A61B 2017/320075

(57) ABSTRACT

An ultrasonic blade includes a body portion defining a top surface, a bottom surface, first and second side surfaces, and a distal face. In one configuration, first and second concave notches are defined in the body portion to form a ridge extending along a portion of a length of the bottom surface. In another configuration, at least one protrusion protrudes from the bottom surface and defines an elongated configuration extending in a length-wise direction along a portion of the bottom surface. In another configuration, the bottom surface includes an angled distal section that is angled towards the top surface in a proximal-to-distal direction such that a distal portion of the body portion tapers in height in the proximal-to-distal direction, and a ridge protrudes from the angled distal section of the bottom surface and extends along at least a portion of a length of the bottom surface.

### FIELD

The present disclosure relates to surgical instruments and, more particularly, to ultrasonic blades and surgical instruments incorporating the same that facilitate performance of multiple different surgical tasks.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to treat tissue. An ultrasonic surgical device may include, for example, an ultrasonic blade and a clamp mechanism to enable clamping of tissue against the blade. Ultrasonic energy transmitted to the blade causes the blade to vibrate at very high frequencies, which allows for heating tissue to treat tissue clamped against or otherwise in contact with the blade. Ultrasonic blades may also be utilized for performing other surgical tasks such as, for example, dissection, scoring, otomies, etc.

Electrosurgical instruments and systems conduct Radio Frequency (RF) energy through tissue to treat tissue. An electrosurgical instrument or system may be configured to conduct bipolar RF energy between oppositely charged electrodes and through tissue, e.g., tissue clamped between the electrodes or otherwise in contact therewith, to treat tissue. Alternatively or additionally, an electrosurgical instrument or system may be configured to deliver monopolar RF energy from an active electrode to tissue in contact with the electrode, with the energy returning via a remote return electrode device to complete the circuit.

### SUMMARY

As used herein, the term “distal” refers to the portion that is described which is further from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is an ultrasonic blade configured for use with an ultrasonic surgical instrument. The ultrasonic blade includes a body portion defining a top surface, a bottom surface, first and second side surfaces, and a distal face. First and second concave notches are defined in the body portion to form a ridge extending along a portion of a length of the bottom surface.

In an aspect of the present disclosure, the first concave notch is defined at an intersection of the first side surface and the bottom surface, and the second concave notch is defined at an intersection of the second side surface and the bottom surface.

In another aspect of the present disclosure, the first concave notch is defined at an intersection of the distal face, the first side surface, and the bottom surface, and the second concave notch is defined at an intersection of the distal face, the second side surface, and the bottom surface.

In yet another aspect of the present disclosure, ridge extends to a distal end of the body portion. Alternatively, the ridge is proximally-spaced from a distal end of the body portion.

In still another aspect of the present disclosure, the first and second concave notches are symmetric with one another, and the ridge is centered relative to the first and second side surfaces of the body portion.

In still yet another aspect of the present disclosure, the ridge is exposed to define a monopolar electrode or coated with an electrically conductive material to define a monopolar electrode.

Another ultrasonic blade configured for use with an ultrasonic surgical instrument and provided in accordance with the present disclosure includes a body portion defining a top surface, a bottom surface, first and second side surfaces, and a distal face, and at least one protrusion protruding from the bottom surface. Each protrusion of the at least one protrusion defines an elongated configuration extending in a length-wise direction along a portion of the bottom surface.

In an aspect of the present disclosure, the at least one protrusion includes a plurality of protrusions spaced apart along a portion of a length of the bottom surface.

In another aspect of the present disclosure, each protrusion of the at least one protrusion includes first and second relatively broad side surfaces and a relatively narrow bottom surface.

In still another aspect of the present disclosure, the relatively narrow bottom surface is exposed to define a monopolar electrode or coated with an electrically conductive material to define a monopolar electrode.

In yet another aspect of the present disclosure, each protrusion of the at least one protrusion is centered relative to the first and second side surfaces of the body portion.

Another ultrasonic blade provided in accordance with the present disclosure and configured for use with an ultrasonic surgical instrument includes a body portion defining a top surface, a bottom surface, first and second side surfaces, and a distal face. The bottom surface includes an angled distal section that is angled towards the top surface in a proximal-to-distal direction such that a distal portion of the body portion tapers in height in the proximal-to-distal direction. A ridge protrudes from the angled distal section of the bottom surface and extends along at least a portion of a length of the bottom surface.

In an aspect of the present disclosure, the ridge defines a variable height along a length thereof.

In another aspect of the present disclosure, the ridge defines an increasing height in a distal-to-proximal direction along at least a portion of a length of the ridge. In such aspects, a slope of the ridge height increase may be substantially complementary to a slope of the angled distal section of the bottom surface.

In still another aspect of the present disclosure, the ridge does not extend beyond the angled distal section of the bottom surface.

In yet another aspect of the present disclosure, the ridge extends the length of the bottom surface.

In still yet another aspect of the present disclosure, the ridge is exposed to define a monopolar electrode or coated with an electrically conductive material to define a monopolar electrode.

In another aspect of the present disclosure, at least a portion of the top surface is exposed to define one electrode of a bipolar configuration or coated with an electrically conductive material to define the one electrode of the bipolar configuration.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a side view of a surgical system provided in accordance with the present disclosure including a surgical instrument, a surgical generator, and, in aspects, a return electrode device;

FIG. 2 is perspective view of another surgical system provided in accordance with the present disclosure including a surgical instrument incorporating an ultrasonic generator, a power source, and, in aspects, an electrosurgical generator therein;

FIG. 3 is a schematic illustration of a robotic surgical system provided in accordance with the present disclosure;

FIG. 4 is a longitudinal, cross-sectional view of a distal end portion of the surgical instrument of FIG. 1;

FIG. 5A is a transverse, cross-sectional view of the end effector assembly of the surgical instrument of FIG. 1;

FIG. 5B is a transverse, cross-sectional view of another configuration of the end effector assembly of the surgical instrument of FIG. 1;

FIGS. 6A and 6B are side, partial schematic views of the end effector assembly of the surgical instrument of FIG. 1 in bipolar and monopolar configurations, respectively;

FIGS. 7A and 7B are perspective and bottom views, respectively, of an ultrasonic blade provided in accordance with aspects of the present disclosure and configured for use with the surgical instrument of FIG. 1;

FIGS. 8A and 8B are bottom and side views, respectively, of another ultrasonic blade provided in accordance with aspects of the present disclosure and configured for use with the surgical instrument of FIG. 1;

FIGS. 9A and 9B are perspective and side views, respectively, of yet another ultrasonic blade provided in accordance with aspects of the present disclosure and configured for use with the surgical instrument of FIG. 1;

FIGS. 10A and 10B are perspective and side views, respectively, of still another ultrasonic blade provided in accordance with aspects of the present disclosure and configured for use with the surgical instrument of FIG. 1;

FIGS. 11A and 11B are perspective and side views, respectively, of still yet another ultrasonic blade provided in accordance with aspects of the present disclosure and configured for use with the surgical instrument of FIG. 1;

FIGS. 12A and 12B are perspective views of further ultrasonic blades provided in accordance with aspects of the present disclosure and configured for use with the surgical instrument of FIG. 1; and

FIG. 13 is a side, partial schematic view illustrating electrical connections to the ultrasonic blade of FIGS. 12A and 12B.

### DETAILED DESCRIPTION

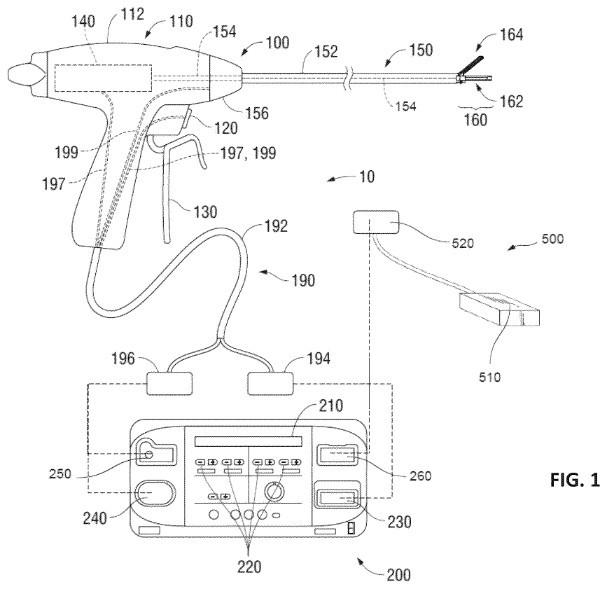

Referring to FIG. 1, a surgical system provided in accordance with aspects of the present disclosure is shown generally identified by reference numeral 10 including a surgical instrument 100, a surgical generator 200, and, in some aspects, a return electrode device 500, e.g., including a return pad 510. Surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200.

Surgical generator 200 includes a display 210, a plurality of user interface features 220, e.g., buttons, touch screens, switches, etc., an ultrasonic plug port 230, a bipolar electrosurgical plug port 240, and active and return monopolar electrosurgical plug ports 250, 260, respectively. As an alternative to plural dedicated ports 230-260, one or more common ports (not shown) may be configured to act as any two or more of ports 230-260.

Surgical instrument 100 may be configured to operate in one or more electrosurgical modes supplying Radio Frequency (RF) energy to tissue to treat tissue, e.g., a monopolar configuration and/or a bipolar configuration, and/or in an ultrasonic mode supplying ultrasonic energy to tissue to treat tissue. Other additional or alternative energy modalities are also contemplated such as, for example, microwave energy, thermal energy, light energy, etc. Surgical generator 200 is configured to produce ultrasonic drive signals for output through ultrasonic plug port 230 to surgical instrument 100 to activate surgical instrument 100 in the ultrasonic mode (where so provided) and/or to provide electrosurgical energy, e.g., RF bipolar energy for output through bipolar electrosurgical plug port 240 and/or RF monopolar energy for output through active monopolar electrosurgical port 250 to surgical instrument 100 to activate surgical instrument 100 in the one or more electrosurgical modes (where so provided). Plug 520 of return electrode device 500 is configured to connect to return monopolar electrosurgical plug port 260 to return monopolar electrosurgical energy from surgical instrument 100 in the monopolar electrosurgical mode.

Continuing with reference to FIG. 1, handle assembly 110 includes a housing 112, an activation button 120, and a clamp trigger 130. Housing 112 is configured to support an ultrasonic transducer 140. Ultrasonic transducer 140 may be permanently engaged within housing 112 or removable therefrom. Ultrasonic transducer 140 includes a piezoelectric stack other suitable ultrasonic transducer components electrically coupled to surgical generator 200, e.g., via one or more of first electrical lead wires 197, to enable communication of ultrasonic drive signals to ultrasonic transducer 140 to drive ultrasonic transducer 140 to produce ultrasonic vibration energy that is transmitted along a waveguide 154 of elongated assembly 150 to blade 162 of end effector assembly 160 of elongated assembly 150, as detailed below. Feedback and/or control signals may likewise be communicated between ultrasonic transducer 140 and surgical generator 200. Ultrasonic transducer 140, more specifically, may include a stack of piezoelectric elements secured, under pre-compression between proximal and distal end masses or a proximal end mass and an ultrasonic horn with first and second electrodes electrically coupled between piezoelectric elements of the stack of piezoelectric elements to enable energization thereof to produce ultrasonic energy. However, other suitable ultrasonic transducer configurations, including plural transducers and/or non-longitudinal, e.g., torsional, transducers are also contemplated.

Activation button 120 is disposed on housing 112 and coupled to or between ultrasonic transducer 140 and/or surgical generator 200, e.g., via one or more of first electrical lead wires 197, to enable activation of ultrasonic transducer 140 in response to depression of activation button 120. In some configurations, activation button 120 may include an ON/OFF switch. In other configurations, activation button 120 may include multiple actuation switches to enable activation from an OFF position to different actuated positions corresponding to different activation settings, e.g., a first actuated position corresponding to a first activation setting (such as a LOW power or tissue sealing setting) and a second actuated position corresponding to a second activation setting (such as a HIGH power or tissue transection setting). In still other configurations, separate activation buttons may be provided, e.g., a first actuation button for activating a first activation setting and a second activation button for activating a second activation setting. Additional activation buttons, sliders, wheels, etc. are also contemplated to enable control of various different activation settings from housing 112.

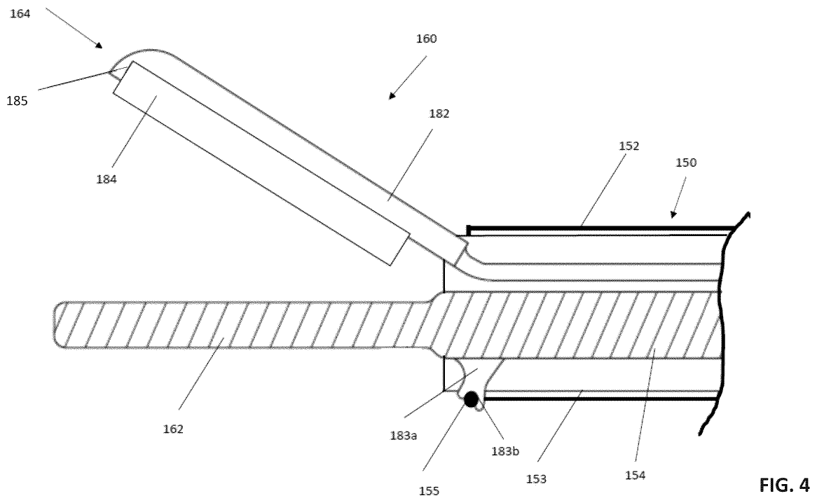

Elongated assembly 150 of surgical instrument 100 includes an outer drive sleeve 152, an inner support sleeve 153 (FIG. 4) disposed within outer drive sleeve 152, a waveguide 154 extending through inner support sleeve 153 (FIG. 4), a drive assembly (not shown), a rotation knob 156, and an end effector assembly 160 including a blade 162 and a jaw member 164. In aspects where only electrosurgical energy is provided and/or other configurations, waveguide 154 and blade 162 may be replaced with a second jaw member (not shown) configured to oppose jaw member 164. Rotation knob 156 is rotatable in either direction to rotate elongated assembly 150 in either direction relative to handle assembly 110. The drive assembly operably couples a proximal portion of outer drive sleeve 152 to clamp trigger 130 of handle assembly 110. A distal portion of outer drive sleeve 152 is operably coupled to jaw member 164 and a distal end of inner support sleeve 153 (FIG. 4) pivotably supports jaw member 164. As such, clamp trigger 130 is selectively actuatable to thereby move outer drive sleeve 152 about inner support sleeve 153 (FIG. 4) to pivot jaw member 164 relative to blade 162 of end effector assembly 160 from a spaced apart position to an approximated position for clamping tissue between jaw member 164 and blade 162. The configuration of outer and inner sleeves 152, 153 (FIG. 4) may be reversed, e.g., wherein outer sleeve 152 is the support sleeve and inner sleeve 153 (FIG. 4) is the drive sleeve. Other suitable drive structures as opposed to a sleeve are also contemplated such as, for example, drive rods, drive cables, drive screws, etc.

Referring still to FIG. 1, the drive assembly may be tuned to provide a jaw clamping force, or jaw clamping force within a jaw clamping force range, to tissue clamped between jaw member 164 and blade 162 or may include a force limiting feature whereby the clamping force applied to tissue clamped between jaw member 164 and blade 162 is limited to a particular jaw clamping force or a jaw clamping force within a jaw clamping force range.

Waveguide 154, as noted above, extends from handle assembly 110 through inner sleeve 153 (FIG. 4). Waveguide 154 includes blade 162 disposed at a distal end thereof. Blade 162 may be integrally formed with waveguide 154, separately formed and subsequently attached (permanently or removably) to waveguide 154, or otherwise operably coupled with waveguide 154. Waveguide 154 and/or blade 162 may be formed from titanium, a titanium alloy, or other suitable electrically conductive material(s), although non-conductive materials are also contemplated. Waveguide 154 includes a proximal connector (not shown), e.g., a threaded male connector, configured for engagement, e.g., threaded engagement within a threaded female receiver, of ultrasonic transducer 140 such that ultrasonic motion produced by ultrasonic transducer 140 is transmitted along waveguide 154 to blade 162 for treating tissue clamped between blade 162 and jaw member 164 or positioned adjacent to blade 162.

Cable assembly 190 of surgical instrument 100 includes a cable 192, an ultrasonic plug 194, and an electrosurgical plug 196. Ultrasonic plug 194 is configured for connection with ultrasonic plug port 230 of surgical generator 200 while electrosurgical plug 196 is configured for connection with bipolar electrosurgical plug port 240 of surgical generator 200 and/or active monopolar electrosurgical plug port 250 of surgical generator 200. In configurations where generator 200 includes a common port, cable assembly 190 may include a common plug (not shown) configured to act as both the ultrasonic plug 194 and the electrosurgical plug 196. In configurations where surgical instrument 100 is only configured for ultrasonic operation, electrosurgical plug 196 and associated components are omitted.

Plural first electrical lead wires 197 electrically coupled to ultrasonic plug 194 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of activation button 120 in an ultrasonic mode. In addition, plural second electrical lead wires 199 are electrically coupled to electrosurgical plug 196 and extend through cable 192 into handle assembly 110. In bipolar configurations, separate second electrical lead wires 199 are electrically coupled to waveguide 154 and jaw member 164 (and/or different portions of jaw member 164) such that bipolar electrosurgical energy may be conducted between blade 162 and jaw member 164 (and/or between different portions of jaw member 164). In monopolar configurations, a second electrical lead wire 199 is electrically coupled to waveguide 154 such that monopolar electrosurgical energy may be supplied to tissue from blade 162. Alternatively or additionally, a second electrical lead wire 199 may electrically couple to jaw member 164 in the monopolar configuration to enable monopolar electrosurgical energy to be supplied to tissue from jaw member 164. In configurations where both bipolar and monopolar functionality are enabled, one or more of the second electrical lead wires 199 may be used for both the delivery of bipolar energy and monopolar energy; alternatively, bipolar and monopolar energy delivery may be provided by separate second electrical lead wires 199. One or more other second electrical lead wires 199 is electrically coupled to activation button 120 to enable the selective supply of electrosurgical energy from surgical generator 200 to waveguide 154 and/or jaw member 164 upon activation of activation button 120 in an electrosurgical mode(s).

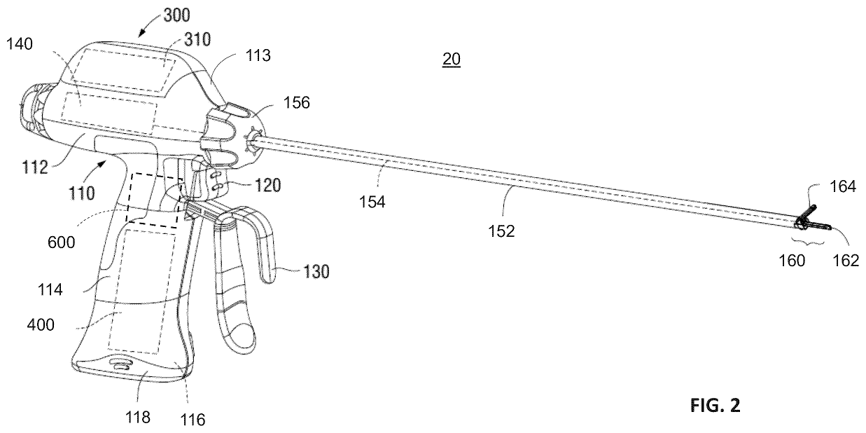

As an alternative to a remote generator 200, surgical system 10 may be at least partially cordless in that it incorporates an ultrasonic generator, an electrosurgical generator, and/or a power source, e.g., a battery, thereon or therein. In this manner, the connections from surgical instrument 100 to external devices, e.g., generator(s) and/or power source(s), are reduced or eliminated. More specifically, with reference to FIG. 2, another surgical system in accordance with the present disclosure is shown illustrated as a surgical instrument 20 supporting an ultrasonic generator 310, a power source (e.g., battery assembly 400), and, in aspects, an electrosurgical generator 600 thereon or therein. Surgical instrument 20 is similar to surgical instrument 100 (FIG. 1) and may include any of the features thereof except as explicitly contradicted below. Accordingly, only differences between surgical instrument 20 and surgical instrument 100 (FIG. 1) are described in detail below while similarities are omitted or summarily described.

Housing 112 of surgical instrument 20 includes a body portion 113 and a fixed handle portion 114 depending from body portion 113. Body portion 113 of housing 112 is configured to support an ultrasonic transducer and generator assembly (“TAG”) 300 including ultrasonic generator 310 and ultrasonic transducer 140. TAG 300 may be permanently engaged with body portion 113 of housing 112 or removable therefrom.

Fixed handle portion 114 of housing 112 defines a compartment 116 configured to receive battery assembly 400 and electrosurgical generator 600 and a door 118 configured to enclose compartment 116. An electrical connection assembly (not shown) is disposed within housing 112 and serves to electrically couple activation button 120, ultrasonic generator 310 of TAG 300, and battery assembly 400 with one another when TAG 300 is supported on or in body portion 113 of housing 112 and battery assembly 400 is disposed within compartment 116 of fixed handle portion 114 of housing 112, thus enabling activation of surgical instrument 20 in an ultrasonic mode in response to appropriate actuation of activation button 120. Further, the electrical connection assembly or a different electrical connection assembly disposed within housing 112 serves to electrically couple activation button 120, electrosurgical generator 600, battery assembly 400, and end effector assembly 160 (e.g., blade 162 and jaw member 164 and/or different portions of jaw member 164) with one another when electrosurgical generator 600 and battery assembly 400 are disposed within compartment 116 of fixed handle portion 114 of housing 112, thus enabling activation of surgical instrument 20 in an electrosurgical mode, e.g., bipolar RF, in response to appropriate actuation of activation button 120. For a monopolar electrosurgical mode, return electrode device 500 (FIG. 1) may be configured to connect to surgical instrument 20 (electrosurgical generator 600 thereof, more specifically), to complete a monopolar circuit through tissue and between surgical instrument 20 (e.g., blade 162 and/or jaw member 164) and return electrode device 500 (FIG. 1).

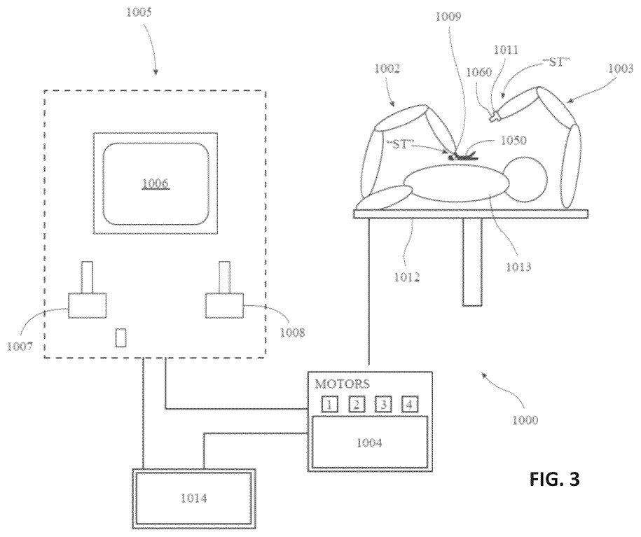

Turning to FIG. 3, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three dimensional images; and manual input devices 1007, 1008, by means of which a person (not shown), for example a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 1050, 1060. One of the surgical tools “ST” may be surgical instrument 100 (FIG. 1), surgical instrument 20 (FIG. 2), or any other suitable surgical instrument 20 configured for use in both an ultrasonic mode and one or more electrosurgical (bipolar and/or monopolar) modes, wherein manual actuation features, e.g., actuation button 120 (FIG. 1), clamp lever 130 (FIG. 1), etc., are replaced with robotic inputs. In such configurations, robotic surgical system 1000 may include or be configured to connect to an ultrasonic generator, an electrosurgical generator, and/or a power source. The other surgical tool “ST” may include any other suitable surgical instrument, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Referring to FIGS. 4-6B, end effector assembly 160 of surgical instrument 100 of surgical system 10 (FIG. 1) is detailed, although the aspects and features of end effector assembly 160 may similarly apply, to the extent consistent, to surgical instrument 20 (FIG. 2) and/or any other suitable surgical instrument or system. End effector assembly 160, as noted above, includes blade 162 and jaw member 164. Blade 162 may define a linear configuration, may define a curved configuration, or may define any other suitable configuration, e.g., straight and/or curved surfaces, portions, and/or sections; one or more convex and/or concave surfaces, portions, and/or sections; etc. With respect to curved configurations, blade 162, more specifically, may be curved in any direction relative to jaw member 164, for example, such that the distal tip of blade 162 is curved towards jaw member 164, away from jaw member 164, or laterally (in either direction) relative to jaw member 164. Further, blade 162 may be formed to include multiple curves in similar directions, multiple curves in different directions within a single plane, and/or multiple curves in different directions in different planes. In addition, blade 162 may additionally or alternatively be formed to include any suitable features, e.g., a tapered configuration, various different cross-sectional configurations along its length, cut outs, indents, edges, protrusions, straight surfaces, curved surfaces, angled surfaces, wide edges, narrow edges, and/or other features.

Blade 162 may define a polygonal, rounded polygonal, or any other suitable cross-sectional configuration(s). Waveguide 154 or at least the portion of waveguide 154 proximally adjacent blade 162, may define a cylindrical shaped configuration. Plural tapered surfaces (not shown) may interconnect the cylindrically shaped waveguide 154 with the polygonal (rounded edge polygonal, or other suitable shape) configuration of blade 162 to define smooth transitions between the body of waveguide 154 and blade 162.

Blade 162 may be wholly or selectively coated with a suitable material, e.g., a non-stick material, an electrically insulative material, an electrically conductive material, combinations thereof, etc. Suitable coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; ElectroBond® coating available from Surface Solutions Group of Chicago, IL, USA; or other suitable coatings and/or methods of applying coatings.

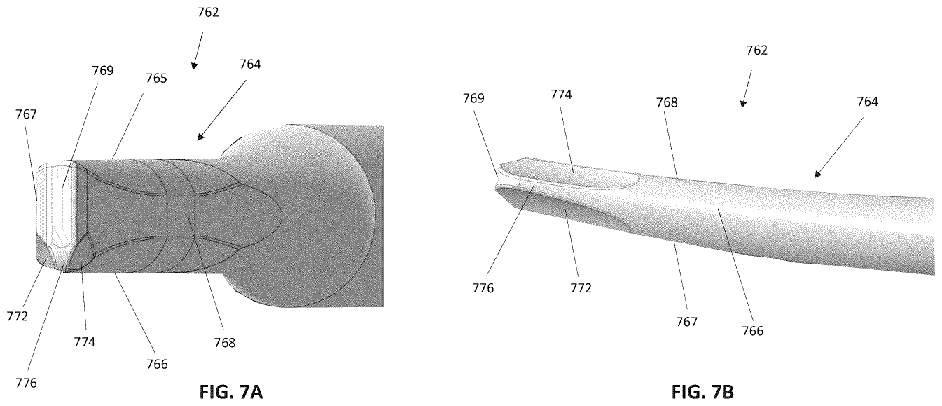

Various different configurations of ultrasonic blades provided in accordance with the present disclosure and suitable for use as blade 162 are detailed below with reference to FIGS. 7A-14.

Continuing with reference to FIGS. 4-6B, blade 162, as noted above, in addition to receiving ultrasonic energy transmitted along waveguide 154 from ultrasonic transducer 140 (FIG. 1), may be adapted to connect to generator 200 (FIG. 1) to enable the supply of RF energy to blade 162 for conduction to tissue in contact therewith. In bipolar configurations, RF energy is conducted between blade 162 and jaw member 164 (or between portions of jaw member 164 and/or blade 162) and through tissue disposed therebetween to treat tissue. In monopolar configurations, RF energy is conducted from blade 162, serving as the active electrode, to tissue in contact therewith and is ultimately returned to generator 200 (FIG. 1) via return electrode device 500 (FIG. 1), serving as the passive or return electrode.

Jaw member 164 of end effector assembly 160 includes more rigid structural body 182 and more compliant jaw liner 184. Structural body 182 may be formed from an electrically conductive material, e.g., stainless steel, and/or may include electrically conductive portions. Structural body 182 includes a pair of proximal flanges 183a that are pivotably coupled to the inner support sleeve 153 via receipt of pivot bosses (not shown) of proximal flanges 183a within corresponding openings (not shown) defined within the inner support sleeve 153 and operably coupled with outer drive sleeve 152 via a drive pin 155 secured relative to outer drive sleeve 152 and pivotably received within apertures 183b defined within proximal flanges 183a. As such, sliding of outer drive sleeve 152 about inner support sleeve 153 pivots jaw member 164 relative to blade 162 from a spaced apart position to an approximated position to clamp tissue between jaw liner 184 of jaw member 164 and blade 162.

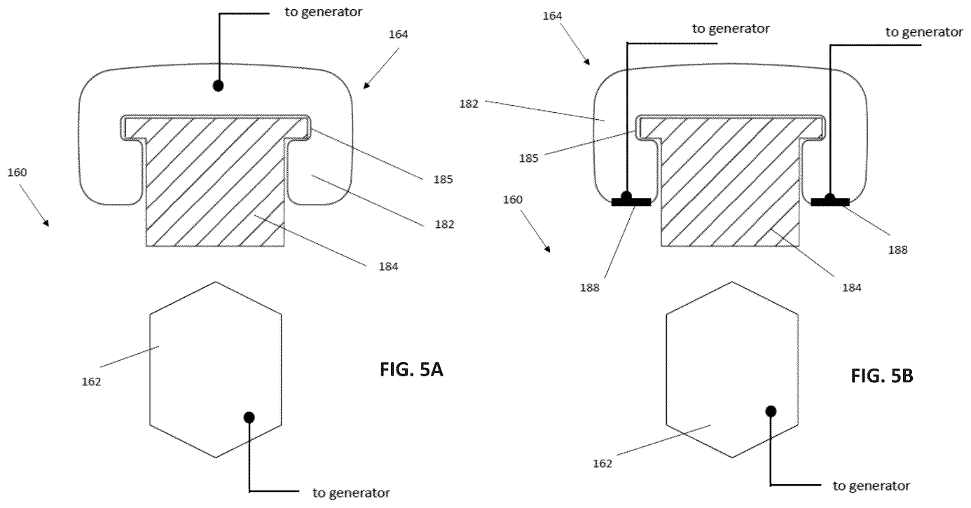

With reference to FIG. 5A, structural body 182 may be adapted to connect to a source of electrosurgical energy, e.g., generator 200 (FIG. 1), and, in a bipolar electrosurgical mode, is charged to a different potential as compared to blade 162 to enable the conduction of bipolar electrosurgical (e.g., RF) energy through tissue clamped therebetween, to treat the tissue. In a monopolar electrosurgical mode, structural body 182 may be un-energized, may be charged to the same potential as compared to blade 162 (thus both defining the active electrode), or may be energized while blade 162 is not energized (wherein structural body 182 defines the active electrode). In either monopolar configuration, energy is returned to generator 200 (FIG. 1) via return electrode device 500 (FIG. 1), which serves as the passive or return electrode.

Referring to FIG. 5B, as an alternative to the entirety of structural body 182 of jaw member 164 being connected to generator 200 (FIG. 1), the structural body may be formed from or embedded at least partially in an insulative material, e.g., an overmolded plastic, or a conductive material coated or otherwise treated to be non-conductive, e.g., PEO-coated titanium, ceramic-coating steel, etc. In such configurations, electrically conductive surfaces 188, e.g., in the form of plates, may be disposed on (e.g., bonded to, deposited onto, mechanically engaged with, etc.) or captured by the insulative material (e.g., overmolded plastic) to define electrodes on either side of jaw liner 184 on the blade facing side of jaw member 164. The electrically conductive surfaces 188, in such aspects, are connected to generator 200 (FIG. 1) and may be energized for use in bipolar and/or monopolar configurations, e.g., energized to the same potential as one another and/or blade 162 and/or different potentials as one another and/or blade 162. In aspects, electrically conductive surfaces 188 are disposed at additional or alternative locations on jaw member 164, e.g., along either or both sides thereof, along a back surface thereof, etc.

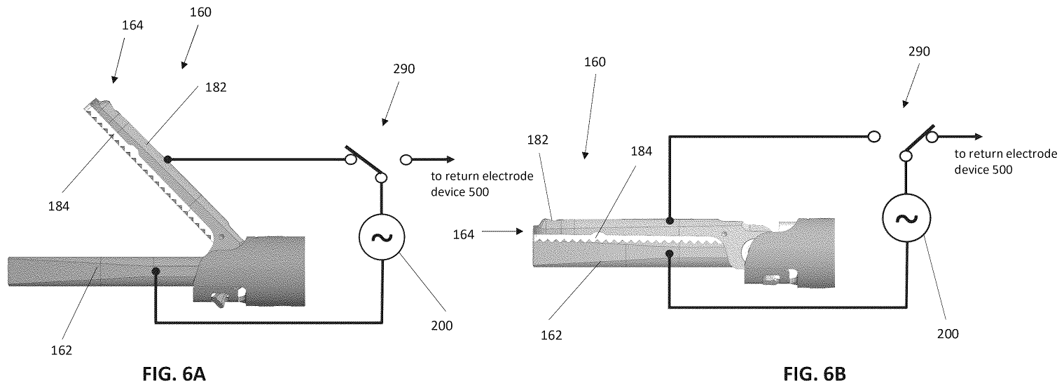

With reference to FIGS. 6A and 6B, in aspects having both monopolar and bipolar configurations, a switch 290 may be provided to selectively connect either jaw member 164 (e.g., structural body 182 or electrically conductive surfaces 188 (see FIGS. 5A and 5B, respectively)) or return electrode device 500 with the generator 200 to enable the return of energy from blade 162, Switch 290 may be manually switched by a button or other user interface feature actuatable by a user (e.g., disposed on handle assembly 110 (FIG. 1) or generator 200 (FIG. 1)) to switch between monopolar and bipolar modes of operation. Alternatively, switch 290 may be automatically switched such as, for example, based on the position of jaw member 164. That is, with jaw member 164 disposed in the spaced apart position (or not sufficiently approximated), switch 290 may connect return electrode device 500 with the generator 200, corresponding to the monopolar mode of operation. On the other hand, with jaw member 164 disposed in the approximated position (or sufficiently approximated), switch 290 may connect jaw member 164 with the generator 200, corresponding to the bipolar mode of operation. In such a configuration, switch may be disposed at end effector assembly 160, e.g., to be mechanically switched under urging from jaw member 164 or drive sleeve 152 (FIG. 4), or may be disposed at handle assembly 110 (FIG. 1), e.g., to be mechanically switched under urging from clamp trigger 130 (FIG. 1) or the drive assembly (not shown).

Again referring to FIGS. 4-6B, jaw liner 184 is retained within a cavity 185 defined within structural body 182. Jaw liner 184 is fabricated from an electrically insulative, compliant material such as, for example, polytetrafluoroethylene (PTFE), although other suitable materials, including conductive materials, partially conductive and partially non-conductive materials, Positive Temperature Coefficient (PTC) materials, Negative Temperature Coefficient (NTC) materials, combinations of the above and/or other materials, etc. are also contemplated. The compliance of jaw liner 184 enables blade 162 to vibrate while in contact with jaw liner 184 without damaging components of ultrasonic surgical instrument 100 (FIG. 1) and without compromising the hold on tissue clamped between jaw member 164 and blade 162. As an alternative to jaw member 164 and, more specifically, jaw liner 184 thereof opposing blade 162 in the approximated position of jaw member 164, jaw member 164 (including jaw liner 184) may be utilized in an end effector assembly having an opposing jaw member (not shown), e.g., for transmission of energy (electrosurgical, thermal, microwave, light, ultrasonic, etc.) to tissue to seal and/or cut tissue grasped therebetween. In such configurations, the opposing jaw member (not shown) may include a thermal cutting element configured to thermally cut tissue and positioned to oppose jaw liner 184 (wherein jaw liner 184 functions as the insulative member of the jaw member), for example, as described in Patent Application Pub. US 2021/0186587, the entire contents of which are hereby incorporated herein by reference.

Jaw liner 184, in aspects, extends from structural body 182 towards blade 162 to inhibit contact between structural body 182 and blade 162 in the approximated position of jaw member 164. The insulation of jaw liner 184 maintains electrical isolation between blade 162 and structural body 182 of jaw member 164, thereby inhibiting shorting. In aspects, jaw liner 184 includes grasping features, e.g., grasping teeth (see FIGS. 6A and 6B), to facilitate grasping and holding tissue against blade 162 in the approximated position of jaw member 164.

Turning to FIGS. 7A-13, various different configurations of ultrasonic blades provided in accordance with the present disclosure and suitable for use as blade 162 (FIG. 1) are detailed below. The ultrasonic blade detailed below enable multi-function use such as, for example, to facilitate tissue sealing, tissue dissection, tissue back scoring, and/or otomy formation, etc., either in an ultrasonic mode, an electrosurgical mode (monopolar or bipolar), or a combined mode utilizing both ultrasonic and electrosurgical (monopolar or bipolar) energy. The ultrasonic blades detailed below with reference to FIGS. 7A-13 may be configured similar to and include any of the above-detailed features of ultrasonic blade 162 (FIG. 1) except as explicitly contradicted below; thus, the following description will focus on the differences between the various ultrasonic blades and ultrasonic blade 162 (FIG. 1), while similarities are summarily described or omitted entirely.

Referring to FIGS. 7A and 7B, ultrasonic blade 762 is shown generally including a body portion 764 having a top surface 765, a bottom surface 766, first and second side surfaces 767, 768, respectively, and a distal face 769. Body portion 764 may be curved in a direction towards one of the side surfaces 767, 768 and away from the other side surface 767, 768. Body portion 764 may also taper in a proximal-to-distal direction in width.

Top surface 765 of body portion 764 of blade 762 is configured to oppose jaw member 164 (FIG. 1) and forms the tissue-contacting surface against which tissue is clamped between jaw member 164 (FIG. 1) and body portion 764 of blade 762 in the approximated position of jaw member 164 (FIG. 1). Top surface 765 may be curved, may include angled surface portions that meet at an apex (or plateau), or may define any other suitable configuration. Although top surface 765 is primarily utilized to facilitate treating, e.g., sealing and/or dissecting, clamped tissue, top surface 765 may also be utilized to treat tissue in an open-jaw configuration; that is, without tissue clamped against top surface 765.

Bottom surface 766 of body portion 764 of blade 762 is positioned opposite top surface 765 and may be utilized to treat tissue dynamically, e.g., via moving blade 762 relative to the tissue to be treated, although static tissue treatment is also contemplated. Bottom surface 766 may be curved, may include angled surface portions that meet at an apex (or plateau), or may define any other suitable configuration.

Side surfaces 767, 768 of body portion 764 of blade 762 may be substantially symmetrical with one another (aside from any asymmetry introduced via the curvature of body portion 764 itself). Side surfaces 767, 768 may be substantially planar (aside from any curvature introduced via the curvature of body portion 764 itself).

Distal face 769 of body portion 764 of blade 762 may be substantially planar although convex configurations or angled surfaces meeting at an apex are also contemplated.

Curved transitions may be defined at the intersections of top surface 765 and side surfaces 767, 768 and at the intersections of bottom surface 766 and side surfaces 767, 768, although angled, chamfered, or other suitable intersections are also contemplated. Likewise, curved or angled surfaces may define the transitions between top surface 765, bottom surface 766, and/or side surfaces 767, 768 with distal face 769.

Body portion 764 of blade 762 further defines a concave notch 772, 774 on each side thereof at each of the bottom corners thereof, e.g., at the three-way intersection between bottom surface 766, side surface 767, and distal face 769 and at the three-way intersection between bottom surface 766, side surface 768, and distal face 769. Notches 772, 774 are symmetrical with one another and cooperate to define a ridge 776 extending to a distal end of body portion 764 of blade 762 on the bottom side thereof. Due to the symmetrical nature of notches 772, 774, ridge 776 is centered relative to side surfaces 767, 768 of body portion 764. Ridge 776 may define a plateaued surface, a curved surface, or may include angled surface portions that meet at an apex. Regardless of the particular configuration of ridge 776, the formation of ridge 776 via notches 772, 774 enables the relatively narrow ridge 776 to be utilized to apply increased pressure to tissue to treat tissue, e.g., via back scoring, without tissue contacting or with decreased pressure applied to tissue via the remainder of body portion 764 of blade 762. The depth of notches 772, 774 and, thus, the height ridge 776 protrudes from the surfaces of notches 772, 774 may be selected to facilitate such tissue treatment.

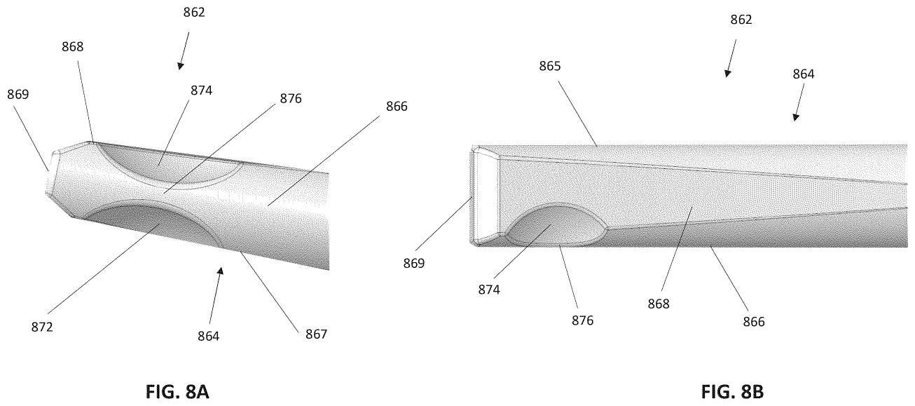

With reference to FIGS. 8A and 8B, another ultrasonic blade 862 is shown similar to ultrasonic blade 762 (FIGS. 7A and 7B) except as explicitly contradicted below. Ultrasonic blade 862 including a body portion 864 having a top surface 865, a bottom surface 866, first and second side surfaces 867, 868, respectively, and a distal face 869.

Body portion 864 of blade 862 further defines a concave notch 872, 874 on each side thereof at the intersection between bottom surface 866 and side surface 867 and at the intersection between bottom surface 866 and side surface 868. Notches 872, 874 differ from notches 772, 774 (FIGS. 7A and 7B) in that notches 872, 874 are proximally-spaced from distal face 869 and the distal end of body portion 864. Notches 872, 874 cooperate to define a ridge 876 extending longitudinally along a portion of body portion 864 of blade 862 on the bottom side thereof. Ridge 876 is proximally-spaced from distal face 869 and the distal end of body portion 864 such that the distal end of body portion 864 defines a more-blunt configuration as compared with the distal end of body portion 764 (FIGS. 7A and 7B). Further, notches 872, 874 facilitate centering of tissue (e.g., on ridge 876) for treatment with ridge 876, e.g., via back scoring as noted above.

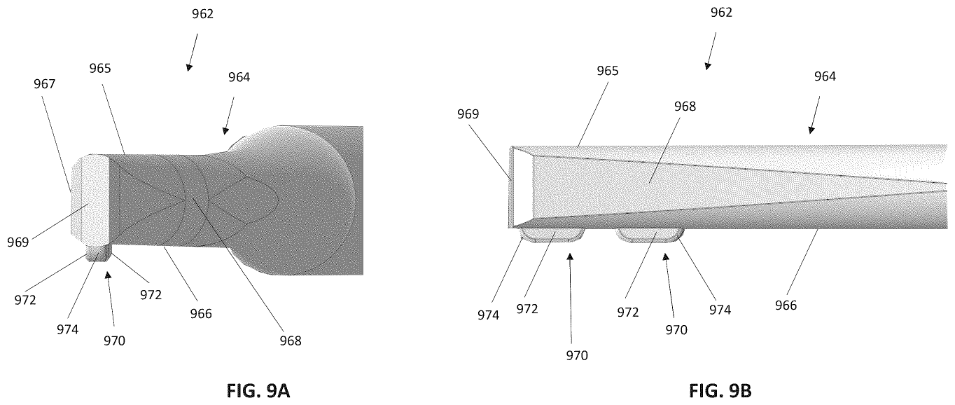

Turning to FIGS. 9A and 9B, another ultrasonic blade 962 is shown generally including a body portion 964 having a top surface 965, a bottom surface 966, first and second side surfaces 967, 968, respectively, and a distal face 969. Body portion 964 may be configured similar to body portion 764 of blade 762 (FIGS. 7A and 7B) except that, rather than defining concave notches that form a ridge therebetween, body portion 964 of blade 962 includes one or more protrusions 970 protruding substantially normally from bottom surface 966. Each protrusion 970 may define a relatively narrow configuration having a pair of relatively broad side surfaces 972 and a relatively narrow bottom surface 974 extending between the side surfaces 972. Where multiple protrusions 970 are provided, the protrusions 970 may be aligned and spaced apart along a portion of a length of body portion 964. A distal-most protrusion 970, in aspects, may overlap a portion of distal face 969, or may be proximally spaced from distal face 969. Protrusions 970 may define any suitable shape configurations such as, for example, rectangular, rounded, triangular, other polygonal, etc. With respect to polygonal configurations, the angles thereof may be rounded, chamfered, or otherwise configured to reduce sharp points. If multiple protrusions 970 are provided, such protrusions 970 may be similar or different from one another. Further, the one or more protrusions 970 may be centered relative to side surfaces 967, 968 or offset relative thereto.

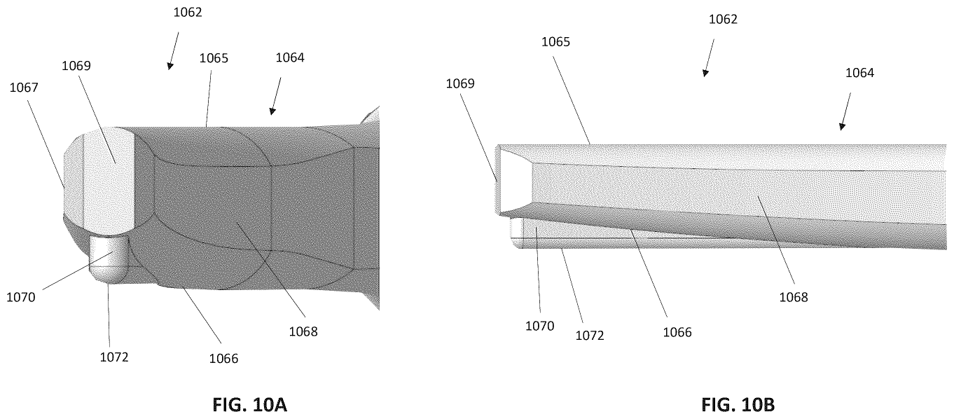

FIGS. 10A and 10B illustrate still another ultrasonic blade 1062 including a body portion 1064 having a top surface 1065, a bottom surface 1066, first and second side surfaces 1067, 1068, respectively, and a distal face 1069. Body portion 1064 may be configured similar to body portion 764 of blade 762 (FIGS. 7A and 7B) except as contradicted below.

Body portion 1064 of blade 1062 tapers in height in a proximal-to-distal direction; however, this taper is not symmetric. Rather, the taper is defined on the bottom side of body portion 1064 such that bottom surface 1066 is angled towards top surface 1065 in a proximal-to-distal direction. Bottom surface 1066 need not be planar to define this angle but can also define a curvature or multiple angled sections that cooperate to achieve this taper of body portion 1064. Top surface 1065 may extend substantially parallel relative to a longitudinal axis defined through blade 1062.

A ridge 1070 protrudes from bottom surface 1066 of body portion 1064 of blade 1062 and extends longitudinally along at least a portion of the length of the tapered section of body portion 1064. Ridge 1070 may be centered on bottom surface 1066 and may define a height that tapers in a distal-to-proximal direction of similar slope (e.g., complementarily to) the taper of body portion 1064 such that, despite the taper of body portion 1064, ridge 1070 extends substantially parallel relative to a longitudinal axis defined through blade 1062. In aspects, ridge 1070 does not protrude downwardly beyond the envelope defined by the un-tapered section of body portion 1064 of blade 1062. In aspects, ridge 1070 protrudes to the downward extent of the envelope defined by the un-tapered section of body portion 1064 of blade 1062. Thus, in such aspects, the overall height of blade 1062, taking into account the tapered section of body portion 1064 and ridge 1070, is substantially uniform along the length of blade 1062, although non-uniform configurations and/or configurations wherein ridge 1070 protrudes beyond the envelope defined by the un-tapered section of body portion 1064 of blade 1062 are also contemplated. Ridge 1070 facilitates balance of blade 1062 when ultrasonically activated.

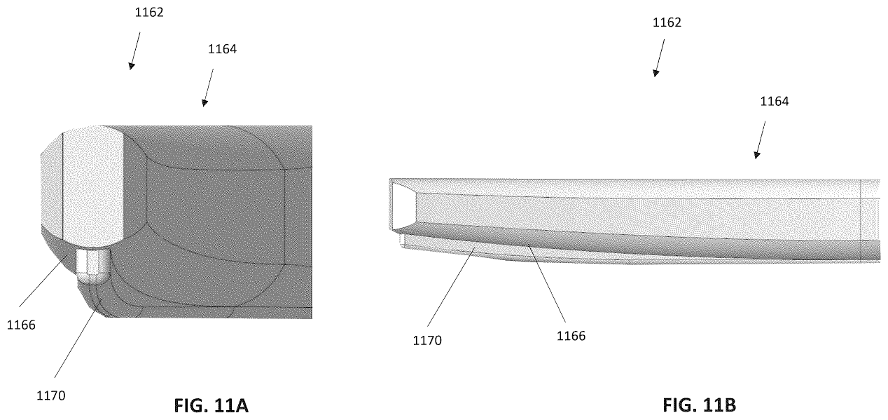

FIGS. 11A and 11B illustrate yet another ultrasonic blade 1162 similar to blade 1062 (FIGS. 10A and 10B) except that, rather than maintaining a substantially uniform overall height of the blade (due to the height taper of the body portion 1064 and the equal height increase of the ridge in the proximal-to-distal direction), as with blade 1062 (FIGS. 10A and 10B), the increase in height of ridge 1170 in the proximal-to-distal direction does not match the taper of bottom surface 1166 to thereby asymmetrically taper the height of blade 1162 in the proximal-to-distal direction. Further, ridge 1170 may extend substantially the entire length of blade 1162. Ridge 1170 may define a first substantially uniform height along the un-tapered portion of blade 1162 and may define a second substantially uniform height along the tapered portion of blade 1162 that is different from the first substantially uniform height. Alternatively, ridge 1170 may define a tapered, curved, angled, or other suitable variable second height (in the proximal-to-distal or distal-to-proximal direction) along the tapered portion of blade 1162. Ridge 1170 facilitates balance of blade 1162 when ultrasonically activated.

With general reference to FIGS. 7A-11B, in aspects providing combined ultrasonic and electrosurgical functionality, any of ultrasonic blades 762-1162 may be configured to connect to a source of electrosurgical energy in a bipolar and/or monopolar configuration. The entire blade 762-1162 may thus serve as an electrode in an electrosurgical (bipolar and/or monopolar) configuration. Alternatively, ultrasonic blades 762-1162 may be partially coated with an insulative material such that the electrode is defined as the exposed portion(s) of the ultrasonic blade 762-1162. Additionally or alternatively, ultrasonic blades 762-1162 may be selectively coated with a conductive material (with an insulative layer disposed therebetween, in aspects) that is connected to a source of electrosurgical energy to serve as the electrode. With respect to either selectively exposed portions of ultrasonic blades 762-1162 to define the electrode(s) or selective conductive coatings that define the electrode(s), at least a portion of the upper surfaces of ultrasonic blades 762-1162 may include the electrode(s), e.g., to define a bipolar configuration itself or in conjunction with the jaw member. Further, at least a portion of the ridges and/or protrusions of ultrasonic blades 762-1162 may include the electrode(s), e.g., to define a monopolar configuration. For such monopolar configurations, monopolar energy and/or combined ultrasonic and monopolar energy may be utilized, in conjunction with the structural configuration of ultrasonic blades 762-1162, to facilitate back scoring, otomy formation, etc.

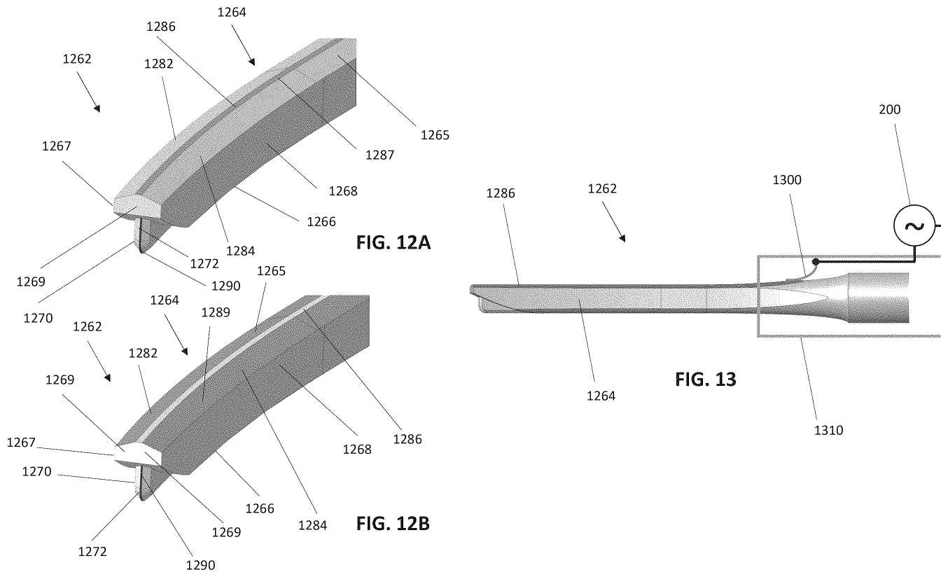

With reference to FIGS. 12A and 12B, another ultrasonic blade 1262 includes a body portion 1264 having a top surface 1265, a bottom surface 1266, first and second side surfaces 1267, 1268, respectively, and a distal face 1269. Body portion 1264 may be configured similar to body portion 1064 of blade 1062 (FIGS. 10A and 10B) except as contradicted below.

A distal portion of body portion 1264 of blade 1262 tapers in height in a proximal-to-distal direction as a result of the upward angling of bottom surface 1266 towards top surface 1265 in a proximal-to-distal direction. A ridge 1270 protrudes from bottom surface 1266 of body portion 1264 of blade 1262 and extends longitudinally along at least a portion of the length of the tapered distal portion of body portion 1264. The tapered distal portion of body portion 1264 and ridge 1270 may define greater slopes and extend a smaller distance along blade 1262 as compared to that of body portion 1064 of blade 1062 and ridge 1070 thereof (see FIGS. 10A and 10B), although body portion 1264 and ridge 1270 may otherwise include any of the features of body portion 1064 and ridge 1070 (FIGS. 10A and 10B).

Top surface 1265 of body portion 1264 includes a pair of angled surfaces 1282, 1284 interconnected via a ridge 1286 which may be rounded, substantially planar, define an edge, etc. As illustrated in FIG. 12A, ridge 1286 may be coated with an electrically conductive material 1287 (including an insulative material between the electrically conductive material 1287 and blade 1262, in aspects) and connected to a source of electrosurgical energy to function as a monopolar electrode or one electrode in a bipolar configuration. Alternatively, angled surfaces 1282, 1284 may be coated with an insulative material 1289 leaving ridge 1286 exposed to connect to a source of electrosurgical energy to function as a monopolar electrode or one electrode in a bipolar configuration. In any of these configurations, electrosurgical energy may be supplied together with ultrasonic energy from blade 1262 (consecutively, overlapping, or simultaneously) or separately therefrom. Exposing or coating the relatively narrow ridge 1286 so as to form an electrode, in aspects, enables plasma blade monopolar electrosurgical functionality such as described in U.S. Pat. No. 9,592,090, the entire contents of which is hereby incorporated herein by reference, although traditional monopolar configurations are also contemplated.

In addition or as an alternative to exposing or coating the relatively narrow ridge 1286 on top surface 1265, the bottom surface 1272 of ridge 1270 protruding from bottom surface 1266 of body 1264 of blade 1262 may be coated with an electrically conductive material 1290 (including an insulative material between the electrically conductive material 1290 and blade 1262, in aspects) or ridge 1270 may be coated with an insulative material except for bottom surface 1272 thereof such that bottom surface 1272 functions as a monopolar electrode. In aspects, this relatively narrow monopolar electrode may provide plasma blade functionality or may function as a traditional monopolar electrode.

Turning to FIG. 13, in aspects where body portion 1264 of blade 1262 includes an electrically conductive material 1286 that functions as an electrode and extends to a proximal portion of blade 1262 (or in aspects where blade 1262 itself functions as the electrode), a spring contact 1300, e.g., a leaf spring, disposed within the one or more sleeves 1310 (e.g., the support sleeve and drive sleeve) through which the ultrasonic waveguide defining blade 1262 extends is biased into contact with the electrically conductive material 1286 (or the blade 1262 in aspects where blade 1262 itself functions as the electrode) so as to establish electrical connection therewith to enable the supply of electrosurgical energy from the energy source, e.g., generator 200, to the electrically conductive material 1286 (or blade 1262). Other locations for spring contact 1300 and/or multiple spring contacts 1300 where multiple isolated electrically-conductive portions are provided may also be utilized. Alternative electrical connection configurations are also contemplated.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. An ultrasonic blade configured for use with an ultrasonic surgical instrument, the ultrasonic blade comprising:
a body portion defining a top surface, a bottom surface, first and second side surfaces, and a distal face,
wherein first and second concave notches are defined in the body portion to form a ridge extending along a portion of a length of the bottom surface.

2. The ultrasonic blade according to claim 1, wherein the first concave notch is defined at an intersection of the first side surface and the bottom surface, and wherein the second concave notch is defined at an intersection of the second side surface and the bottom surface.

3. The ultrasonic blade according to claim 1, wherein the first concave notch is defined at an intersection of the distal face, the first side surface, and the bottom surface, and wherein the second concave notch is defined at an intersection of the distal face, the second side surface, and the bottom surface.

4. The ultrasonic blade according to claim 1, wherein the ridge extends to a distal end of the body portion.

5. The ultrasonic blade according to claim 1, wherein the ridge is proximally-spaced from a distal end of the body portion.

6. The ultrasonic blade according to claim 1, wherein the first and second concave notches are symmetric with one another and wherein the ridge is centered relative to the first and second side surfaces of the body portion.

7. The ultrasonic blade according to claim 1, wherein the ridge is exposed to define a monopolar electrode or coated with an electrically conductive material to define a monopolar electrode.

8. An ultrasonic blade configured for use with an ultrasonic surgical instrument, the ultrasonic blade comprising:
a body portion defining a top surface, a bottom surface, first and second side surfaces, and a distal face,
wherein at least one protrusion protrudes from the bottom surface, each protrusion of the at least one protrusion defining an elongated configuration extending in a length-wise direction along a portion of the bottom surface.

9. The ultrasonic blade according to claim 8, wherein the at least one protrusion includes a plurality of protrusions spaced apart along a portion of a length of the bottom surface.

10. The ultrasonic blade according to claim 8, wherein each protrusion of the at least one protrusion includes first and second relatively broad side surfaces and a relatively narrow bottom surface.

11. The ultrasonic blade according to claim 10, wherein the relatively narrow bottom surface is exposed to define a monopolar electrode or coated with an electrically conductive material to define a monopolar electrode.

12. The ultrasonic blade according to claim 8, wherein each protrusion of the at least one protrusion is centered relative to the first and second side surfaces of the body portion.

13. An ultrasonic blade configured for use with an ultrasonic surgical instrument, the ultrasonic blade comprising:
a body portion defining a top surface, a bottom surface, first and second side surfaces, and a distal face, wherein the bottom surface includes an angled distal section that is angled towards the top surface in a proximal-to-distal direction such that a distal portion of the body portion tapers in height in the proximal-to-distal direction,
wherein a ridge protrudes from the angled distal section of the bottom surface and extends along at least a portion of a length of the bottom surface.

14. The ultrasonic blade according to claim 13, wherein the ridge defines a variable height along a length thereof.

15. The ultrasonic blade according to claim 13, wherein the ridge defines an increasing height in a distal-to-proximal direction along at least a portion of a length of the ridge.

16. The ultrasonic blade according to claim 15, wherein a slope of the ridge height increase is substantially complementary to a slope of the angled distal section of the bottom surface.

17. The ultrasonic blade according to claim 13, wherein the ridge does not extend beyond the angled distal section of the bottom surface.

18. The ultrasonic blade according to claim 13, wherein the ridge extends the length of the bottom surface.

19. The ultrasonic blade according to claim 13, wherein the ridge is exposed to define a monopolar electrode or coated with an electrically conductive material to define a monopolar electrode.

20. The ultrasonic blade according to claim 13, wherein at least a portion of the top surface is exposed to define one electrode of a bipolar configuration or coated with an electrically conductive material to define the one electrode of the bipolar configuration.
